(19) United States
(12) Patent Application Publication
(10) Pub. No.: US 2023/0338621 A1
(43) Pub. Date: Oct. 26, 2023

(54) SURFACES HAVING LUBRICIOUS OR LOW FRICTION PROPERTIES

(71) Applicant: Evonik Canada Inc., Burlington (CA)

(72) Inventors: Alexandra Piotrowicz, Mississauga (CA); Antonio Cillero Rodrigo, Toronto (CA); Jiaqing Zuo, Mississauga (CA); Jeannette Ho, Toronto (CA)

(73) Assignee: Evonik Canada Inc., Burlington, ON (CA)

(21) Appl. No.: 18/027,860

(22) PCT Filed: Sep. 24, 2021
(86) PCT No.: PCT/CA2021/051331

§ 371 Date: Mar. 22, 2023

Related U.S. Application Data
(60) Provisional application No. 63/083,305, filed on Sep. 25, 2020.

(51) Int. Cl. A61L 29/08; A61L 29/14

(52) U.S. Cl. CPC A61L 29/085; A61L 29/14; A61L 2400/10; A61L 2420/02

(57) ABSTRACT

An article having a low friction surface containing an oligofluorinated additive admixed with a base polymer is disclosed.

### BACKGROUND OF THE INVENTION

### Field of the Invention

The invention relates the use of oligofluorinated additives (also referred to as surface modifying macromolecules or SMMs) for the purposes of modifying surface properties to generate lubricious or low friction surfaces. The additives can be combined with a base polymer to form admixtures used in the manufacture of devices, components, or coatings requiring a low coefficient of friction (CoF).

### Discussion of the Background

Low coefficient of friction or lubricity of the surface of an article is desirable in a variety of applications where facile movement of one surface over another is required for proper or optimal function. This is especially relevant for medical device applications. For example, in devices such as catheters or guidewires, lubricious surfaces enable the use of lower insertion forces, easier tracking through complex vasculature, reduced tissue irritation/damage, and improved patient comfort. Additionally, lubricity may be desirable to facilitate drainage of fluids within medical conduits. Lubricity may be required on all parts of a medical device, or only some parts of the device.

One common method of creating low friction surfaces in medical devices is the application of hydrophilic, or water-loving coatings. These are typically based on hydrogel polymers such as PVP, which can absorb large amounts of water. Coatings, however, can be susceptible to abrasion and delamination, which can lead to loss of functionality and present hazards due to generation of particulates within the body. For example, particulates generated from coatings on intravascular catheters can cause an embolism with devastating consequences for the patient. Coatings can also be difficult to apply to certain substrates, often requiring base coats, tie coats, and cure methods, and may need to be specifically tailored for the coating substrate to ensure that plasticizers or colorants blooming to the surface of substrates do not impact coating adhesion. The use of organic solvents or polymerizable monomers and associated reagents in the coating process can pose toxicity concerns if residual reagents are not fully removed from the final coating. Additionally, coating application requires an equipment investment that is costly, and generates a significant amount of reagent waste.

Another limitation of coatings is that application to all device configurations, for example multiple catheter lumens, can be challenging or not possible. Also, in the case of hydrogel coatings specifically, significant swelling upon absorption of water can increase the coating thickness, which may impact device functionality if dimensional tolerances are important. Certain hydrogel coatings may also be susceptible to drying out quickly, being unable to maintain sufficient hydration after removal from the hydration solution and prior to use, leading to loss of lubricious properties.

Another method to create low friction surfaces on thermoplastic catheter tubing is co-extrusion with very thin PTFE liners, but this process requires significant technical expertise and is therefore expensive. Yet another method is to compound PTFE powders or lubricating oils into thermoplastics used for extrusion. However, these surfaces often require “break-in” in terms of removal of some material from the surface to perform adequately, which is not suitable for all applications. Furthermore, PTFE powders can create bonding issues in post-extrusion processes.

Polymers used to create medical devices, components or coatings requiring low friction properties can be selected from the groups of polyurethanes, silicones, polyamides, polyesters, co-polyesters, polyethers, polyether-block-amide co-polymers, polypropylenes, polyethylenes, polyvinylchlorides, polysulfones, polyetherimides, polycarbonates, polyetheretherketones, ethyl vinyl acetates, and polyolefins, styrenic block copolymers and vulcanized rubbers, among others. The polymers may include other additives such as radiopaque fillers, colorants, processing aids, antimicrobials, or antiseptics, among others.

Santerre U.S. Pat. No. 6,127,507 discloses fluoro-oligomer surface modifiers for polymers and articles made therefrom.

Mullick et al. U.S. Pat. Nos. 8,071,683, 8,178,620 and 8,338,537 report surface modifying macromolecules with high degradation temperatures and uses thereof.

Mullick et al. U.S. Pat. Nos. 8,318,867, 9,751,972, U.S. 2011/0207893, and U.S. 2018/0179327 report thermally stable biuret and isocyanurate based surface modifying macromolecules and uses thereof.

Mullick et al. U.S. 2017/0369646 reports ester-linked surface modifying macromolecules.

Santerre et al. WO 2019/169500 reports carbonate-linked surface modifying macromolecules.

Steedman et al. U.S. 2019/0142317 reports implantable glucose sensors having a biostable surface.

### SUMMARY OF THE INVENTION

In the present invention, oligofluorinated additives (SMMs) are admixed with the base polymer or material composite used in the manufacture of an article, such as a medical device requiring low friction properties wherein the low friction surface includes an oligofluorinated additive admixed with a base polymer.

In a further aspect, the invention features an article having a surface with low friction properties.

A further aspect of the invention is directed to a medical device having a surface with low friction properties.

A further aspect of the invention is directed to a medical catheter having a surface with low friction properties.

A further aspect of the invention is directed to a method of reducing the coefficient of friction of a surface by applying a mixture comprising an oligofluorinated additive and a base polymer, to a surface.

A further aspect of the invention is directed to a method of producing an article by melt extruding or molding a composition comprising an oligofluorinated additive and a base polymer.

These and other aspects of the invention are provided for by an article, having a surface comprising an oligofluorinated additive admixed with a base polymer.

The inventors have discovered that an admixture of an oligofluorinated additive and a base polymer provides for a surface having low friction properties.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 illustrates the coefficient of friction of polyether block amide co-polymer rods with and without a surface modifying additive;

FIG. 2 illustrates the coefficient of friction of polyurethane rods with and without a surface modifying additive;

FIG. 3 illustrates the coefficient of friction of polyurethane rods with and without a surface modifying additive;

FIG. 4 illustrates the coefficient of friction of polyurethane catheter tubing with and without a surface modifying additive;

FIG. 5 illustrates the coefficient of friction of silicone catheter tubing with and without a surface modifying additive;

FIG. 6 illustrates the coefficient of friction of polyurethane films with and without a surface modifying additive.

### DETAILED DESCRIPTION OF THE PREFERRED EMBODIMENTS

### Fluorooligomers (SMMs)

In one aspect the invention provides a fluorooligomer (also referred to as surface modifying macromolecules or SMMs) having a central portion and terminal groups, the central portion including a segmented oligomeric copolymer unit and the terminal groups including α-w-terminal polyfluoro oligomeric groups.

The additives will migrate to all surfaces of the article during manufacturing due to their surface-active properties, creating a unique surface chemistry.

Preferably, the polyfluoro oligomeric group is a perfluoroalkyl group; and the polar hard segment is selected from the group consisting of a urethane, ester, amide, sulfonamide, and carbonate.

The SMM's, according to the invention, are synthesized in a manner that they contain a base polymer compatible segment and terminal hydrophobic fluorine components which are non-compatible with the base polymer. The compatible segment of the SMM is selected to provide an anchor for the SMM within the base polymer substrate upon admixture. While not being bound by theory, it is believed that the fluorine tails are responsible in-part for carrying the SMM to the surface of the admixture, with the fluorine chains exposed out from the surface. The latter process is believed to be driven by the thermodynamic incompatibility of the fluorine tail with the polymer base substrate, as well as the tendency towards establishing a low surface energy at the mixture's surface. When the balance between anchoring and surface migration is achieved, the SMM remains stable at the surface of the polymer, while simultaneously altering surface properties. The utility of the additives of the invention versus other known macromolecular additives, lies in 1) the molecular arrangement of the amphipathic segments in the SMM chain, i.e. two w fluoro-tails, one at each end, with the central portion between them; and 2) the molecular weight of the fluorine tails relative to that of the central portions.

More specifically, compounds of the invention include formulations synthesized using hydrophilic or amphiphilic building blocks incorporated into the backbone of the SMM molecule. These building blocks can include hydrophilic reactive monomers or oligomers, or reactive oligomers having both hydrophilic and hydrophobic segments. Examples of building blocks include oligomeric diols containing polyethylene oxide (PEO) segments alone, or alternating with hydrophobic polyether or siloxane segments.

Suitable fluorooligomers may also be described by the structure of any one of formulae (I), (II), (III), (IV), (V) and (VI) shown below.

(1) Formula (I):

F_{T}—[B-A]ₙ—B—F_{T} (I)
where
(i) A includes polypropylene oxide, polyethylene oxide, polytetramethylene oxide, hydrogenated polybutadiene (e.g., HLBH), polybutadiene (e.g., LBHP), hydrogenated polyisoprene (e.g., HHTPI), poly(diethylene glycol)adipate, (diethylene glycol-ortho phthalic anhydride) polyester, (neopentyl glycol-ortho phthalic anhydride) polyester, (1,6-hexanediol-ortho phthalic anhydride) polyester, poly(hexamethylene carbonate), poly((2,2-dimethyl)-1,3-propylene carbonate), polycarbonate polyol, poly(ethylene-co-butylene), polystyrene, polysiloxane, polydimethylsiloxane, polypropylene glycol-polyethylene glycol block copolymer, polysiloxane-polyethylene glycol block copolymer, polysiloxane-polypropylene glycol block copolymer, or other block copolymers containing block segments selected from the group of polypropylene oxide, polyethylene oxide, polytetramethylene oxide, polysiloxane, and polydimethylsiloxane;
(ii) B is a segment including a urethane; and
(iii) F_{T} is a polyfluoroorgano group, and
(iv) n is an integer from 1 to 10.

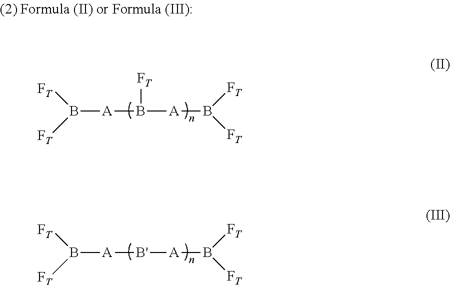

where
(i) A is an oligomeric segment containing an ether linkage, an ester linkage, a carbonate linkage, a polyalkylene. A includes polypropylene oxide, polyethylene oxide, polytetramethylene oxide, hydrogenated polybutadiene (e.g., HLBH), polybutadiene (e.g., LBHP), hydrogenated polyisoprene (e.g., HHTPI), poly(diethylene glycol)adipate, (diethylene glycol-ortho phthalic anhydride) polyester, (neopentyl glycol-ortho phthalic anhydride) polyester, (1,6-hexanediol-ortho phthalic anhydride) polyester, poly(hexamethylene carbonate), poly((2,2-dimethyl)-1,3-propylene carbonate), polycarbonate polyol, poly(ethylene-co-butylene), polystyrene, polysiloxane, polydimethylsiloxane, polypropylene glycol-polyethylene glycol block copolymer, polysiloxane-polyethylene glycol block copolymer, polysiloxane-polypropylene glycol block copolymer, or other block copolymers containing block segments selected from the group of polypropylene oxide, polyethylene oxide, polytetramethylene oxide, polysiloxane, and polydimethylsiloxane;
(ii) B is a segment including an isocyanurate trimer or biuret trimer; B′, when present, is a segment including a urethane;
(iii) each F_{T} is a polyfluoroorgano group; and
(iv) n is an integer between 0 to 10.

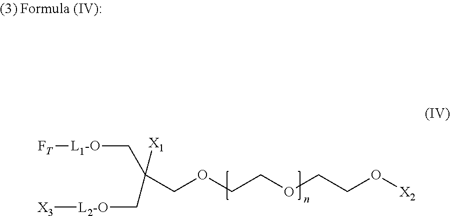

where
(i) each F_{T} is independently a surface-active group selected from polydimethylsiloxanes, hydrocarbons, and polyfluoroorgano groups, and combinations thereof (e.g., each F_{T} is independently a polyfluoroorgano);
(ii) X₁ is H, CH₃, or CH₂CH₃;
(iii) each of X₂ and X₃ is independently H, CH₃, CH₂CH₃, or F_{T};
(iv) each of L₁ and L₂ is independently a bond, an oligomeric linker, or a linker with two terminal carbonyls; and
(v) n is an integer from 5 to 50.

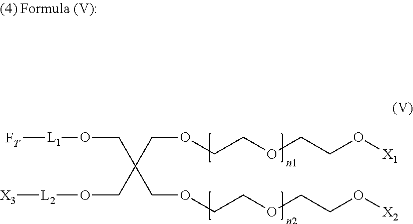

where
(i) each F_{T} is independently a surface-active group (e.g., a polyfluoroorgano, hydroxyl, or polyethylene glycol);
(ii) each of X₁, X₂, and X₃ is independently H, CH₃, CH₂CH₃, or F_{T};
(iii) each of L₁ and L₂ is independently a bond, an oligomeric linker, a linker with two terminal carbonyls, or is formed from a diisocyanate; and
(iv) each of n1 and n2 is independently an integer from 5 to 50.

In one embodiment of Formula (I), A is polypropylene oxide, polyethylene oxide or polytetramethylene oxide.

In another embodiment of Formula (I), A is polypropylene oxide, polyethylene oxide, or polytetramethylene oxide, or a mixture thereof, and has a theoretical molecular weight of from 200 to 3,000 Daltons (e.g., from 500 to 2,000 Daltons, from 1,000 to 2,000 Daltons, or from 1,000 to 3,000 Daltons);

In another embodiment of Formula (I), A is a segment selected from the group consisting of hydrogenated polybutadiene (e.g., HLBH), poly(diethylene glycol)adipate, or (diethylene glycol-ortho phthalic) anhydride polyester, and has a theoretical molecular weight of from 750 to 3,500 Daltons (e.g., from 750 to 2,000 Daltons, from 1,000 to 2,500 Daltons, or from 1,000 to 3,500 Daltons).

In another embodiment of Formula (I), A includes a polypropylene glycol-polyethylene glycol block copolymer (PLN diol), or a block copolymer with a block segment selected from polypropylene oxide, polyethylene oxide, polytetramethylene oxide, or a mixture thereof, and a block segment selected from a polysiloxane or polydimethylsiloxane (C10 diol), where A has a theoretical molecular weight of from 1,000 to 5,000 Daltons (e.g., from 1,000 to 3,000 Daltons, from 2,000 to 5,000 Daltons, or from 2,500 to 5,000 Daltons).

In another embodiment of Formula (II) or (III), A is an oligomeric segment including polyethylene oxide, polypropylene oxide, polytetramethylene oxide, or a mixture thereof, and having a theoretical molecular weight of from 200 to 3,000 Daltons (e.g., from 500 to 2,000 Daltons, from 1,000 to 2,000 Daltons, or from 1,000 to 3,000 Daltons);

In another embodiment of Formula (II) or (III), A includes a polypropylene glycol-polyethylene glycol block copolymer (PLN diol), or a block copolymer with a block segment selected from polypropylene oxide, polyethylene oxide, polytetramethylene oxide, or a mixture thereof, and a block segment selected from polysiloxane or polydimethylsiloxane (C10 diol), where A has a theoretical molecular weight of from 1,000 to 5,000 Daltons (e.g., from 1,000 to 3,000 Daltons, from 2,000 to 5,000 Daltons, or from 2,500 to 5,000 Daltons);

In another embodiment of Formula (II) or (III), A is hydrogenated polybutadiene (e.g., HLBH), a poly(diethylene glycol)adipate, or (diethylene glycol-ortho phthalic) anhydride polyester, and has a theoretical molecular weight of from 750 to 3,500 Daltons (e.g., from 750 to 2,000 Daltons, from 1,000 to 2,500 Daltons, or from 1,000 to 3,500 Daltons).

The SMM of formula (I) can include B formed from a diisocyanate (e.g., 3-isocyanatomethyl-3,5,5-trimethyl-cyclohexylisocyanate; 4,4′-methylene bis(cyclohexyl isocyanate); 4,4′-methylene bis(phenyl isocyanate); toluene-2,4-diisocyanate; m-tetramethylxylene diisocyanate; or hexamethylene diisocyanate). The variable n may be 1 or 2. The medical device of the invention may contain a base polymer and the SMM of formula (I).

In the SMM of formulae (II) or (III), B is formed by reacting a triisocyanate (e.g., hexamethylene diisocyanate (HDI) biuret trimer, isophorone diisocyanate (IPDI) trimer, or hexamethylene diisocyanate (HDI) trimer) with a diol including the oligomeric segment A. The medical device of the invention may contain a base polymer and the SMM of formula (II). The medical device of the invention may contain a base polymer and the SMM of formula (III).

In the SMM of formula (I), B may be a segment formed from 3-isocyanatomethyl-3,5,5-trimethyl-cyclohexylisocyanate; 4,4′-methylene bis(cyclohexyl isocyanate); 4,4′-methylene bis(phenyl isocyanate); toluene-2,4-diisocyanate; m-tetramethylxylene diisocyanate; and hexamethylene diisocyanate. In the SMM of formula (I), segment A can be poly(ethylene oxide). The variable n may be an integer from 1 to 3. The medical device of the invention may contain a base polymer and the SMM of formula (I).

In the SMM of formula (II) or (III), B is a segment formed by reacting a triisocyanate. with a diol of A. The triisocyanate may be hexamethylene diisocyanate (HDI) biuret trimer, isophorone diisocyanate (IPDI) trimer, or hexamethylene diisocyanate (HDI) trimer. In the SMM of formula (II) or (III), segment A can be poly(ethylene oxide). The variable n may be 0, 1, 2, or 3. The medical device of the invention may contain a base polymer and the SMM of formula (II) or (III).

In the SMM of formula (I), B may be a segment formed from 3-isocyanatomethyl-3,5,5-trimethyl-cyclohexylisocyanate; 4,4′-methylene bis(cyclohexyl isocyanate); 4,4′-methylene bis(phenyl isocyanate); toluene-2,4-diisocyanate; m-tetramethylxylene diisocyanate; and hexamethylene diisocyanate. In the SMM of formula (I), segment A can be poly(ethylene oxide)-b-poly(propylene oxide)-b-poly(ethylene oxide). The variable n may be an integer from 1 to 3. The medical device of the invention may contain a base polymer and the SMM of formula (I).

In the SMM of formula (II) or (III), B is a segment formed by reacting a triisocyanate. with a diol of A. The triisocyanate may be hexamethylene diisocyanate (HDI) biuret trimer or hexamethylene diisocyanate (HDI) trimer. In the SMM of formula (II) or (III), segment A can be poly(ethylene oxide)-b-poly(propylene oxide)-b-poly(ethylene oxide). The variable n may be 0, 1, 2, or 3. The medical device of the invention may contain a base polymer and the SMM of formula (II) or (III).

In SMM of formula (I), B is a segment formed from a diisocyanate. The segment A can include polysiloxane-polyethylene glycol block copolymer (e.g., PEG-PDMS-PEG). The segment B may be formed from 3-isocyanatomethyl-3,5,5-trimethyl-cyclohexylisocyanate; 4,4′-methylene bis(cyclohexyl isocyanate); 4,4′-methylene bis(phenyl isocyanate); toluene-2,4-diisocyanate; m-tetramethylxylene diisocyanate; and hexamethylene diisocyanate. The variable n may be 1, 2, or 3. The medical device of the invention may contain a base polymer and the SMM of formula (I).

In the SMM of formula (II) or (III), B is a segment formed by reacting a triisocyanate with a diol of A. The segment A can include polysiloxane-polyethylene glycol block copolymer (e.g., PEG-PDMS-PEG). The triisocyanate may be hexamethylene diisocyanate (HDI) biuret trimer, isophorone diisocyanate (IPDI) trimer, or hexamethylene diisocyanate (HDI) trimer. The variable n may be 0, 1, 2, or 3. The medical device of the invention may contain a base polymer and the SMM of formula (II) or (III).

The SMM of formula (IV) can include a segment L₁ that is an oligomeric linker (e.g., of fewer than 50 repeating units (e.g., from 2 to 40 units, from 2 to 30 units, from 3 to 20 units, or from 3 to 10 units)). In some embodiments of formula (IV), L₂ is an oligomeric linker (e.g., of fewer than 50 repeating units (e.g., from 2 to 40 units, from 2 to 30 units, from 3 to 20 units, or from 3 to 10 units)). In particular embodiments of formula (IV), each of L₁ and L₂ is a bond. In certain embodiments of formula (IV), the SMM includes an oligomeric segment (e.g., in any one of L₁ and L₂) selected from the group consisting of polyurethane, polyurea, polyamide, polyalkylene oxide (e.g., polypropylene oxide, polyethylene oxide, or polytetramethylene oxide), polyester, polylactone, polysilicone, polyethersulfone, polyolefin, polyvinyl derivative, polypeptide, polysaccharide, polysiloxane, polydimethylsiloxane, poly(ethylene-co-butylene), polyisobutylene, and polybutadiene. In some embodiments of formula (IV), the SMM is a compound of formula (IV-A):

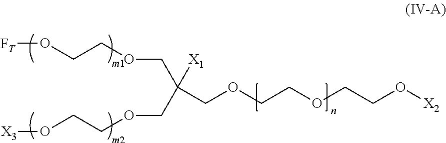

where each of m1 and m2 is independently an integer from 0 to 50. In particular embodiments of formula (IV-A), m1 is 5, 6, 7, 8, 9, or 10 (e.g., m1 is 6). In some embodiments of formula (IV-A), m2 is 5, 6, 7, 8, 9, or 10 (e.g., m2 is 6).

In certain embodiments of formula (IV) or (IV-A), X₂ is F_{T}. In other embodiments, X₂ is CH₃ or CH₂CH₃. In particular embodiments of formula (IV) or (IV-A), X₃ is F_{T}. In other embodiments, each F_{T} is independently a polyfluoroorgano (e.g., a polyfluoroacyl, such as —(O)_{q}—C(═O)]ᵣ(CH₂)ₒ(CF₂)ₚCF₃, in which q is 0, r is 1; o is from 0 to 2; and p is from 0 to 10). In certain embodiments of formula (IV) or (IV-A), n is an integer from 5 to 40 (e.g., from 5 to 20, such as from 5, 6, 7, 8, 9, or 10). In some embodiments of formula (IV) or (IV-A), each F_{T} includes (CF₂)₅CF₃. The medical device of the invention may contain a base polymer and the SMM of formula (IV). The medical device of the invention may contain a base polymer and the SMM of formula (IV-A).

The SMM of formula (V) can include a segment L₁ that is an oligomeric linker (e.g., of fewer than 50 repeating units (e.g., from 2 to 40 units, from 2 to 30 units, from 3 to 20 units, or from 3 to 10 units)). In some embodiments of formula (V), L₂ is an oligomeric linker (e.g., of fewer than 50 repeating units (e.g., from 2 to 40 units, from 2 to 30 units, from 3 to 20 units, or from 3 to 10 units)). In particular embodiments of formula (V), each of L₁ and L₂ is a bond. In certain embodiments of formula (V), the SMM includes an oligomeric segment (e.g., in any one of L₁ and L₂) selected from polyurethane, polyurea, polyamide, polyalkylene oxide (e.g., polypropylene oxide, polyethylene oxide, or polytetramethylene oxide), polyester, polylactone, polysilicone, polyethersulfone, polyolefin, polyvinyl derivative, polypeptide, polysaccharide, polysiloxane, polydimethylsiloxane, poly(ethylene-co-butylene), polyisobutylene, or polybutadiene. In some embodiments of formula (V), the SMM is a compound of formula (V-A):

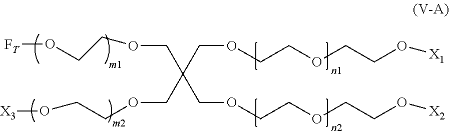

where each of m1 and m2 is independently an integer from 0 to 50. In particular embodiments of formula (V-A), m1 is 5, 6, 7, 8, 9, or 10 (e.g., m1 is 6). In some embodiments of formula (V-A), m2 is 5, 6, 7, 8, 9, or 10 (e.g., m2 is 6).

In certain embodiments of formula (V) or (V-A), X₂ is F_{T}. In other embodiments of formula (V) or (V-A), X₂ is CH₃ or CH₂CH₃. In particular embodiments of formula (V) or (V-A), X₃ is F_{T}. In other embodiments of formula (V) or (V-A), each F_{T} is independently a polyfluoroorgano (e.g., a polyfluoroacyl, such as —(O)_{q}—[C(═O)]ᵣ(CH₂)ₒ(CF₂)ₚCF₃, in which q is 0, r is 1; o is from 0 to 2; and p is from 0 to 10). In some embodiments of formula (V) or (V-A), each F_{T} includes (CF₂)₃CF₃. The medical device of the invention may contain a base polymer and the SMM of formula (V).

The medical device of the invention may contain a base polymer and the SMM of formula (V-A).

For any of the SMMs of the invention formed from a diisocyanate, the diisocyanate may be 3-isocyanatomethyl-3,5,5-trimethyl-cyclohexylisocyanate; 4,4′-methylene bis(cyclohexyl isocyanate) (HMDI); 2,2′-, 2,4′-, and 4,4′-methylene bis(phenyl isocyanate) (MDI); toluene-2,4-diisocyanate; aromatic aliphatic isocyanate, such 1,2-, 1,3-, and 1,4-xylene diisocyanate; meta-tetramethylxylene diisocyanate (m-TMXDI); para-tetramethylxylene diisocyanate (p-TMXDI); hexamethylene diisocyanate (HDI); ethylene diisocyanate; propylene-1,2-diisocyanate; tetramethylene diisocyanate; tetramethylene-1,4-diisocyanate; octamethylene diisocyanate; decamethylene diisocyanate; 2,2,4-trimethylhexamethylene diisocyanate; 2,4,4-trimethylhexamethylene diisocyanate; dodecane-1,12-diisocyanate; dicyclohexylmethane diisocyanate; cyclobutane-1,3-diisocyanate; cyclohexane-1,2-diisocyanate; cyclohexane-1,3-diisocyanate; cyclohexane-1,4-diisocyanate; methyl-cyclohexylene diisocyanate (HTDI); 2,4-dimethylcyclohexane diisocyanate; 2,6-dimethylcyclohexane diisocyanate; 4,4′-dicyclohexyl diisocyanate; 2,4′-dicyclohexyl diisocyanate; 1,3,5-cyclohexane triisocyanate; isocyanatomethylcyclohexane isocyanate; 1-isocyanato-3,3,5-trimethyl-5-isocyanatomethylcyclohexane; isocyanatoethylcyclohexane isocyanate; bis(isocyanatomethyl)-cyclohexane; 4,4′-bis(isocyanatomethyl)dicyclohexane; 2,4′-bis(isocyanatomethyl)dicyclohexane; isophoronediisocyanate (IPDI); 2,4-hexahydrotoluene diisocyanate; 2,6-hexahydrotoluene diisocyanate; 3,3′-dimethyl-4,4′-biphenylene diisocyanate (TODD; polymeric MDI; carbodiimide-modified liquid 4,4′-diphenylmethane diisocyanate; para-phenylene diisocyanate (PPDI); meta-phenylene diisocyanate (MPDI); naphthylene-1,5-diisocyanate; 2,4′-, 4,4′-, or 2,2′-biphenyl diisocyanate; polyphenyl polymethylene polyisocyanate (PMDI); mixtures of MDI and PMDI; mixtures of PMDI and TDI; dimerized uretdione of any isocyanate described herein, such as uretdione of toluene diisocyanate, uretdione of hexamethylene diisocyanate, or a mixture thereof; or a substituted or isomeric mixture thereof.

For any of the SMMs of the invention formed from an isocyanate trimer, the isocyanate trimer can be hexamethylene diisocyanate (HDI) biuret or trimer, isophorone diisocyanate (IPDI) trimer, hexamethylene diisocyanate (HDI) trimer; 2,2,4-trimethyl-1,6-hexane diisocyanate (TMDI) trimer; a trimerized isocyanurate of any isocyanates described herein, such as isocyanurate of toluene diisocyanate, trimer of diphenylmethane diisocyanate, trimer of tetramethylxylene diisocyanate, or a mixture thereof; a trimerized biuret of any isocyanates described herein; modified isocyanates derived from the above diisocyanates; or a substituted or isomeric mixture thereof.

The SMM can include the group F_{T} that is a polyfluoroorgano group having a theoretical molecular weight of from 100 Da to 1,500 Da. For example, F_{T} may be CF₃(CF₂)ᵣ(CH₂CH₂)ₚ—wherein p is 0 or 1, r is 2-20, and CF₃(CF₂)ₛ(CH₂CH₂O)ₓ, where X is from 0 to 10 and s is from 1 to 20. Alternatively, F_{T} may be CHₘF₍₃₋ₘ₎(CF₂)ᵣCH₂CH₂— or CHₘF₍₃₋ₘ₎(CF₂)ₛ(CH₂CH₂O)ₓ—, where m is 0, 1, 2, or 3; X is an integer from 0 to 10; r is an integer from 2 to 20; and s is an integer from 1 to 20. In certain embodiments, F_{T} is 1H,1H,2H,2H-perfluoro-1-decanol; 1H,1H,2H,2H-perfluoro-1-octanol; 11H,1H,5H-perfluoro-1-pentanol; or 1H,1H-perfluoro-1-butanol, or a mixture thereof. In particular embodiments, F_{T} is (CF₃)(CF₂)₅CH₂CH₂O—, (CF₃)(CF₂)₇CH₂CH₂O—, (CF₃)(CF₂)₅CH₂CH₂O—, CHF₂(CF₂)₃CH₂O—, (CF₃)(CF₂)₂CH₂O—, or (CF₃)(CF₂)₅—. In still other embodiments the polyfluoroalkyl group is (CF₃)(CF₂)₅—, e.g., where the polyfluoroalkyl group is bonded to a carbonyl of an ester group. In certain embodiments, polyfluoroorgano is —(O)_{q}[C(═O)]ᵣ—(CH₂)ₒ(CF₂)ₚCF₃, in which q is 0 and r is 1, or q is 1 and r is 0; o is from 0 to 2; and p is from 0 to 10.

Also suitable is a compound of formula (VI):

F_{T}—OC(O)O—B—OC(O)O-[A-OC(O)O—B]ₙ—OC(O)O—F_{T} (VI)

in which
(i) A comprises a soft segment and is covalently bound to B via a carbonate linkage;
(ii) B comprises a polyalkylene oxide or a moiety described by the formula:

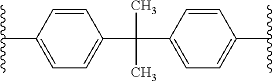 [SMILES: *c1ccc(C(C)(C)c2ccc(*)cc2)cc1]

and is covalently bound to A via a carbonate linkage; and
(iii) F_{T} is a surface-active group comprising a polyfluoroorgano group, wherein F_{T} is covalently bound to B via a carbonate linkage; and
(iv) n is an integer from 1 to 10.

Also suitable is a compound of formula (I) wherein,
(i) A includes

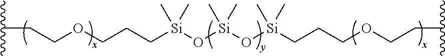

(ii) B is a segment including a urethane formed from 4,4′-methylene bis(cyclohexyl isocyanate); (iii) F_{T} is a polyfluoroorgano group; and (iv) x is an integer from 8 to 12, y is an integer from 6-9, and n is an integer from 1 to 10. In particular embodiments, n is 1 or 2.

The invention features a compound of formula (I):
wherein,
(i) A includes a segment having the formula:

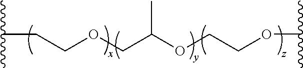

wherein said segment has a MW of 7,000 to 9,000 Da, includes from 75% to 85% (w/w) polyethylene oxide, and includes 15% to 25% (w/w) polypropylene oxide; (ii) B is a segment including a urethane formed from 4,4′-methylene bis(cyclohexyl isocyanate); (iii) F_{T} is a polyfluoroorgano group; and (iv) n is an integer from 1 to 10. In particular embodiments, n is 1 or 2. In some embodiments, A has an average MW of about 8,000 Da and includes about 80% (w/w) polyethylene oxide and about 20% (w/w) polypropylene oxide.

The invention features a compound of formula (II):
wherein,
(i) A includes a segment having the formula:

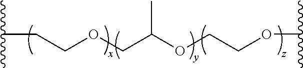

wherein said segment has a MW of 7,000 to 9,000 Da, includes from 75% to 85% (w/w) polyethylene oxide, and includes 15% to 25% (w/w) polypropylene oxide; (ii) B is a segment including an isocyanurate trimer or biuret trimer formed from isophorone diisocyanate (IPDI) trimer; (iii) F_{T} is a polyfluoroorgano group; and (iv) n is an integer from 0 to 10.

In an embodiment of any of the above compounds, F_{T} is selected from the group consisting of radicals of the general formula CHₘF₍₃₋ₘ₎(CF₂)ᵣCH₂CH₂— and CHₘF₍₃₋ₘ₎(CF₂)ₛ(CH₂CH₂O)ₓ—, wherein m is 0, 1, 2, or 3; X is an integer between 1-10; r is an integer between 2-20; and s is an integer between 1-20. In certain embodiments, m is 0 or 1.

Specific SMMs of the invention include compounds with the formulas

Compound 1

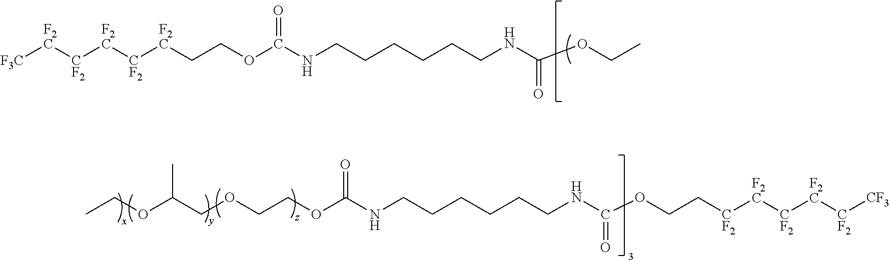

having a PEG content of 50 wt. %; (x+z):y ratio approximately 1.38; Mn:˜1,900 g/mol
Compound 2
having a PEG content of 50 wt. %; (x+z):y ratio approximately 1.38; Mn:˜1,900 g/mol
Compound 3
having a PEG content of 40 wt. %; (x+z):y ratio approximately 0.88; Mn:˜2,900 g/mol
Compound 4

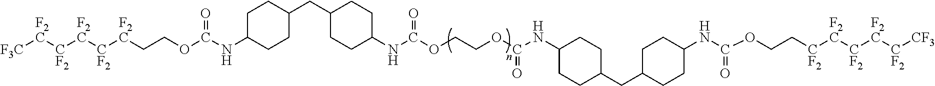

where n˜46
Compound 5

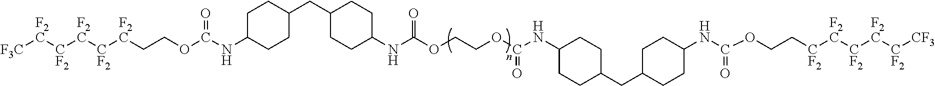

where n˜9
Compound 6

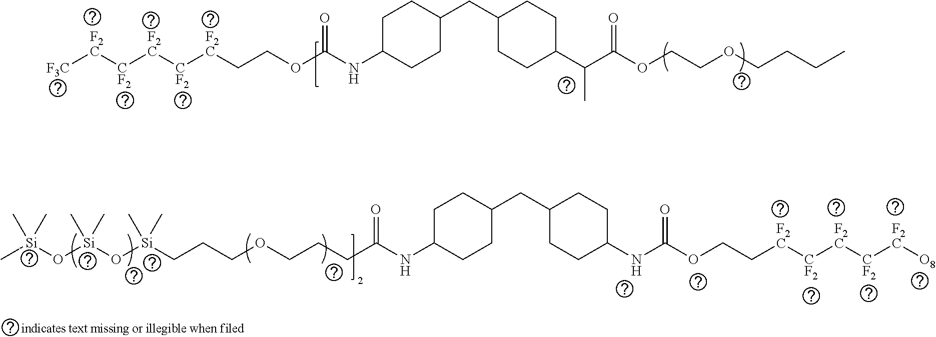

Where n=20 to 28
and Compound 7

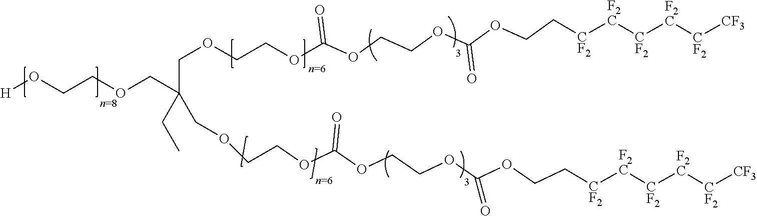

Suitable fluorooligomers are described in U.S. Pat. Nos. 6,127,507, 8,071,683, 8,178,620, 8,338,537, 8,318,867, 9,751,972, U.S. 2011/0207893, U.S. 2018/0179327, U.S. 2017/0369646, WO 2019/169500 and U.S. 2019/0142317, the descriptions of which are hereby incorporated by reference.

SMM compounds may have a theoretical molecular weight of greater than or equal to 500 Da and less than or equal to 20 kDa. Non-limiting examples of SMMs include those having a theoretical molecular weight of from 500 to 10,000 Da, from 500 to 9,000 Da, from 500 to 5,000 Da, from 1,000 to 10,000 Da, from 1,000 to 6,000 Da, or from 1,500 to 8,000 Da. One of skill in the art will recognize that these structural formulae represent idealized theoretical structures. Specifically, the segments are reacted in specific stoichiometries to furnish an oligofluorinated additive as a distribution of molecules having varying ratios of segments. Accordingly, the variable n in formulae (I), (II), (III), (IV), (V) and (VI) indicates the theoretical stoichiometry of the segments.

Methods for preparing suitable SMM compounds are known to those of ordinary skill in the art, without undue experimentation.

### Base Polymers

Examples of typical base polymers of use in admixture with aforesaid SMM according to the invention include a polyurethane (PU), a silicone, a polyamide (PA), a polyester, a co-polyester, a polyether, a polyether-block-amide co-polymer (PEBA), a polyetherimide, a polycarbonate, a polyetheretherketone (PEEK), an ethyl vinyl acetate (EVA) a polypropylene (PP), a polyethylene (PE), a polyvinylchloride (PVC), a polyvinyl alcohol (PVA), a polyvinylpyrrolidone (PVP), a polyacrylamide (PAAM), a polyethylene oxide (PEO), a poly(ethylene oxide)-b-poly(propylene oxide)-b-poly(ethylene oxide), a poly(hydroxyethylmethacrylate) (polyHEMA), polyethylene terephthalate (PET), a polybutylene terephthalate (PBT), a polymethylmethacrylate (PMMA), a polysulfone, a styrenic block copolymer, a vulcanized rubber, a polyolefin, a cyclic olefin polymer (COP), cyclic olefin copolymer (COC), a cellulosic polymer, or a copolymer or blend thereof.

The base polymer has a theoretical molecular weight of greater than or equal to 20 kDa (e.g., greater than or equal to 50 kDa, greater than or equal to 75 kDa, greater than or equal to 100 kDa, greater than or equal to 150 kDa, or greater than 200 kDa).

In further embodiments, the base polymer is a thermoplastic.

The base polymer may contain suitable additives known to those of ordinary skill in the art, such as fillers, colorants, pigments, stabilizers, antioxidants, plasticizers, reinforcing agents, impact modifiers, blowing agents, curing agents, flame retardants, antistatic agents, conductive agents, processing aids, antimicrobials, antiseptics, antibiotics or other functional additives. The additives may be organic or inorganic in nature. Example fillers include radioopaque fillers such as barium sulphate, bismuth subcarbonate, bismuth trioxide, or tungsten. Example plasticizers include bis (2-ethylhexyl)phthalate (DEHP), di(2-ethylhexyl) terephthalate (DEHT), or trioctyltrimellitate (TOTM) used to plasticize PVC resins. Example antimicrobial, antiseptic or antibiotic agents include triclosan, silver sulfadiazine, chlorohexidine, rifampin or clindamycin.

In a preferred aspect the invention provides a composition comprising in admixture of a base polymer and a compatible surface-modifying macromolecule in a surface-modifying enhancing amount.

The amount of fluorooligomer/base polymer is 0.05-15% w/w, preferably 0.1-12% w/w, more preferably 0.5-10% w/w even more preferably 1-5% w/w of fluoro-oligomer in the base polymer.

In one embodiment, the oligofluorinated additive is of formula (I) and said base polymer is selected from the group consisting of a polyurethane, a silicone and a polyether-block-amide co-polymer (PEBA).

In another embodiment, the oligofluorinated additive is of formula (IV) and the base polymer is a polyurethane.

In another embodiment, the oligofluorinated additive of the invention reduces the coefficient of friction of the base polymer, to a greater amount than when the oligofluorinated additive Compound 8 with structure shown below

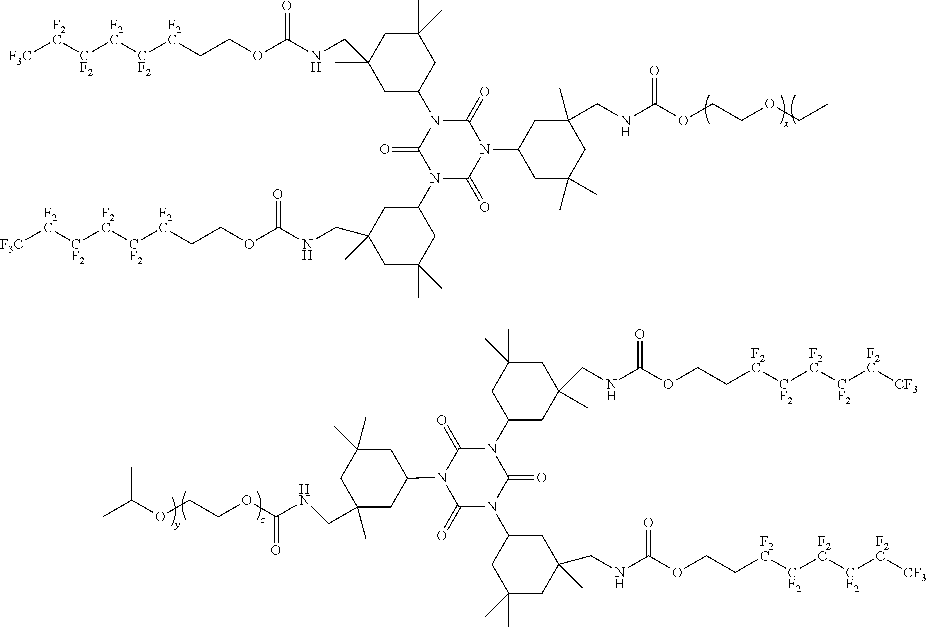

having a PEG content of 50 wt. %, (x+z):y ratio approximately 1.38, Mn:˜1,900 g/mol, is added to Carbothane 85A polyurethane.

Devices

Medical devices requiring low friction properties can include catheters, shunts, surgical cannulae, guidewires, stents, grafts, stent-grafts, endoprostheses, angioplasty balloons, insertion sheaths, introducers, stylets, implantable biosensors, contraceptive devices, breast implants, scaffolds, tympanostomy tubes, ophthalmic devices, contact lenses, IOLs, corneal implants, endotracheal tubes, tracheostomy tubes, endoscopes, syringes, medical blood or fluid transfer tubing, 3D-printed implants, orthopaedic implants, prosthetic implants, implantable pacemaker and defibrillator leads, LVAD drivelines, structural heart implants, wound retractors, endoscopes, vena cava filters, device valves and manifolds, vascular closure devices, and embolic protection devices, among others. Catheters specifically can include vascular catheters, drainage catheters, neurovascular catheters, infusion catheters, parenteral feeding catheters, stroke therapy catheters, urological catheters, peritoneal dialysis catheter, support catheter, diagnostic catheters, atherectomy catheters, electrophysiology catheters, microcatheters, angioplasty catheters, mechanical thrombectomy catheter, aspiration catheter, imaging catheter, and delivery catheters, among others.

The device manufacturing processes relating to this invention can include melt processes such as extrusion or molding, or solution processes such as film casting, solution spinning, electrospinning, dip coating, spray coating, and 3D printing, among others, and techniques known to those of ordinary skill in the art without undue experimentation.

The migration of the SMMs to the device surface can be confirmed using standard analytical methods such as X-ray Photoelectron Spectroscopy, and the surface fluorine can range from between 1 to 50 atomic percent. The relative hydrophilicity of the modified surfaces can be determined by water contact angle analysis.

Coefficient of friction (CoF) can be measured by a number of different test methods, including the ASTM D1894-14 test, pinch test, tortuous path test and various custom tribological methods. The pinch test is particularly common for testing lubricity of medical devices such as catheter tubing. In this test, a test article such as catheter tubing is connected at one end to an instrument capable of measuring pulling force. The sample is submerged in a fluid of interest and clamped between two pads of a specified substrate at a specified force. The sample is then pulled at a specified speed and the force required to move the sample is recorded. The CoF is calculated by dividing the force required to pull the sample by the force exerted on the sample by the clamps. Repeated test cycles can be performed in sequence on the same sample to evaluate the durability of the surface modification.

In a preferred embodiment the pinch test is performed using a Harland FTS 6000 tester, whereby a rod or tubing sample 15 cm in length is mounted in the device, submerged in water at room temperature, and clamped between silicone friction pads (60A durometer) with a fixed force of 200 g, then the sample is pulled at a constant speed of 1 cm/s over a travel distance of 10 cm, and the force required to pull the sample is recorded. The average pull force divided by the clamp force is used to calculate the kinetic CoF.

In another preferred embodiment, the pinch test is repeated on the same sample (e.g. 5 to 25 times) to evaluate coefficient of friction and durability of the surface modification.

The devices modified with SMM additives of the invention can have surfaces with improved lubricity as demonstrated by reduced CoF relative to same unmodified surfaces under either dry or wet conditions. In certain embodiments, reductions in CoF on SMM-modified devices of greater than 50% can be achieved, as tested in the pinch test utilizing water or PBS as the conditioning media, 100-500 g as the clamp force, silicone or PTFE pads as the clamp substrate, 1 cm/min as the pulling speed, and travel distance of 10 cm.

The SMM additives of the invention can be used to reduce the CoF in various resins used to manufacture a device, component, or coating, including but not limited to the aforementioned resins. The additives can be used to reduce the CoF in any conceivable devices or applications where it is important to minimize friction between surfaces, and in particular in medical devices that can be inserted into or moved within the body. The SMM additives can function to reduce the CoF for single-use applications, or applications where the surfaces may be exposed to repeated frictional forces.

Having generally described this invention, a further understanding can be obtained by reference to certain specific examples which are provided herein for purposes of illustration only and are not intended to be limiting unless otherwise specified.

### EXAMPLES

### Example 1: Reduction of Coefficient of Friction in PEBA Rods

Polyether block amide co-polymer (PEBA) prototypes containing 2-4 wt. % compounds of the invention were prepared using a laboratory micro compounder. Specifically, PEBAX 2533 from Arkema was dried in a vacuum oven at 65° C. for 6 h. The resin was blended with different compounds of the invention using a 15 mL twin-screw micro-compounder in batch mode, with a cycle time of 3 min (after resin load) and melt temperature of ˜160° C. The blend was extruded into rods approximately ˜3.5 mm in diameter by opening the mixing chamber valve to release the molten polymer. The resulting rods were quenched in a water bath and air dried.

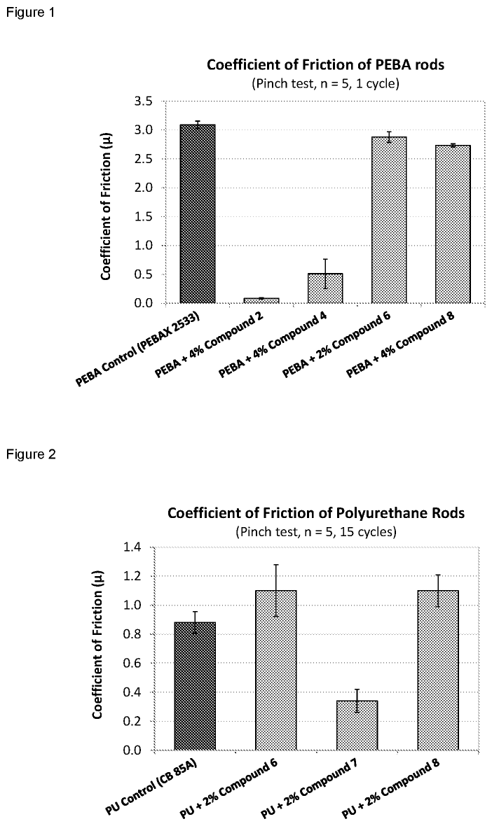

The coefficient of friction (CoF) of the extruded prototypes was measured by means of a pinch test using a Harland FTS 6000 tester. A rod sample 15 cm in length was mounted in the device, submerged in water at room temperature, and clamped between silicone friction pads (60A durometer) with a fixed force of 200 g. The sample was then pulled at a constant speed of 1 cm/s over a travel distance of 10 cm, and the force required to pull the sample was recorded. The average pull force divided by the clamp force was used to calculate the kinetic CoF. Five independent samples of unmodified PEBAX control and SMM-modified prototypes were tested. The average CoF is presented in FIG. 1 with error bars representing standard deviation. Two of the tested SMM formulations, Compound 2 and Compound 4, were found to reduce the CoF of PEBA prototypes by >30% (specifically by 97% and 83% respectively).

### Example 2: Reduction of Coefficient of Friction in Polyurethane Rods

Polyurethane prototypes containing 2 wt. % compounds of the invention were prepared using a laboratory micro-compounder as described in Example 1. Carbothane 3585A from Lubrizol was dried in a vacuum oven at 65° C. for 4 h prior to processing, and a melt temperature of ˜230° C. was used for blending and extrusion of the polyurethane resin with SMM. Resulting rod prototypes were ˜3.3 mm in diameter.

The coefficient of friction (CoF) of the extruded prototypes was measured by means of a pinch test using a Harland FTS 6000 tester as described in Example 1. A clamp force of 510 g was used, and 5 independent samples of unmodified polyurethane control and SMM-modified prototypes were tested. The pinch test was repeated 15 times on each sample to confirm maintained reductions over repeat test cycles. The average CoF across 5 samples and 15 cycles is presented in FIG. 2, with error bars representing standard deviation. One of the tested SMM formulations, Compound 7, was found to reduce CoF of the polyurethane prototypes by >30% (specifically by 61%).

### Example 3: Reduction of Coefficient of Friction in Polyurethane Rods

Polyurethane prototypes containing 2 wt. % compounds of the invention were prepared using a laboratory micro-compounder as described in Example 1. Carbothane 3585A from Lubrizol was dried in a vacuum oven at 65° C. for 4 h prior to processing, and a melt temperature of ˜230° C. was used for blending and extrusion of the polyurethane resin with SMM. Resulting rod prototypes were ˜3.3 mm in diameter.

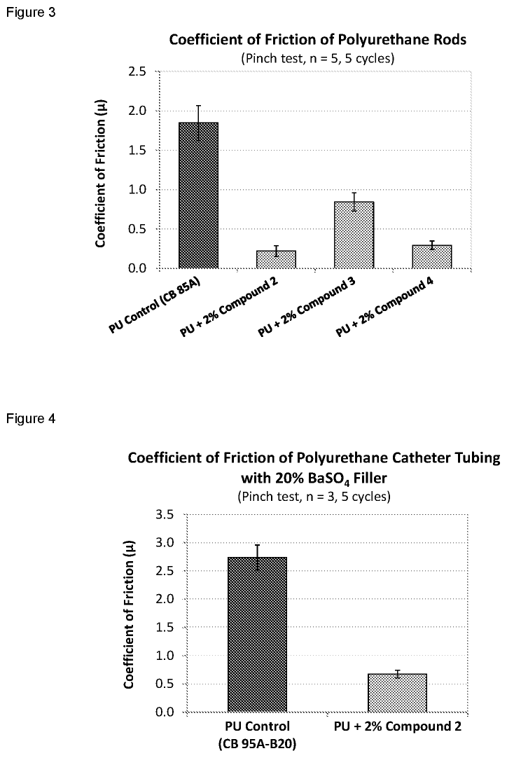

The coefficient of friction (CoF) of the extruded prototypes was measured by means of a pinch test using a Harland FTS 6000 tester as described in Example 1. A clamp force of 200 g was used, and 5 independent samples of unmodified polyurethane control and SMM-modified prototypes were tested. The pinch test was repeated 5 times on each sample to confirm maintained reductions over repeat test cycles. The average CoF across 5 samples and 5 cycles is presented in FIG. 3, with error bars representing standard deviation.

All three of the tested SMM formulations, Compound 2, Compound 4 and Compound 3 were found to reduce the CoF of the polyurethane prototypes by >30% (specifically by 88%, 84% and 54% respectively).

### Example 4: Reduction of Coefficient of Friction in Polyurethane Catheter Tubing

Polyurethane catheter tubing containing 20% barium sulfate radiopaque filler and 2 wt. % Compound 2 of the invention was prepared by first compounding the SMM into Carbothane 3595A-B20 resin from Lubrizol using commercial compounding processes. Both virgin polyurethane resin and compounded resin with SMM were then extruded into 10F tubing using commercial tubing extrusion processes.

The coefficient of friction (CoF) of the extruded tubing was measured by means of a pinch test using a Harland FTS 6000 tester as described in Example 1. A clamp force of 200 g was used, and 3 independent samples of unmodified polyurethane control tubing and SMM-modified tubing were tested. The pinch test was repeated 5 times on each sample to confirm maintained reductions over repeat test cycles. The average CoF across 3 samples and 5 cycles is presented in FIG. 4, with error bars representing standard deviation. The tested SMM formulation, Compound 2, was found to reduce the CoF of polyurethane tubing with 20% barium sulfate by 75%.

### Example 5: Reduction of Coefficient of Friction in Silicone Catheter Tubing

Silicone catheter tubing containing 4 wt. % compound Compound 6 of the invention was prepared by blending the SMM with Silastic Q7-4750 Silicone elastomer from Dow Corning using a 2-roll mill, followed by cold extrusion into 15.5 Fr tubing and curing as per standard silicone tubing extrusion methods. Control silicone tubing was manufactured using the same methods but without SMM added.

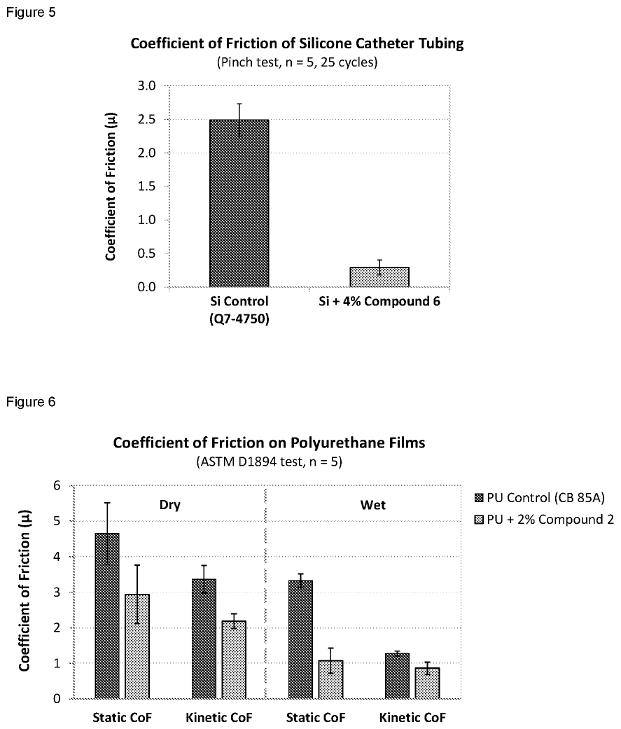

The coefficient of friction of the extruded prototypes was measured by means of a pinch test using a Harland FTS 6000 tester as described in Example 1. A clamp force of 200 g was used, and 5 independent samples of unmodified silicone control tubing and SMM-modified tubing were tested. The pinch test was repeated 25 times on each sample to confirm maintained reductions over repeat test cycles. The average CoF across 5 samples and 25 cycles is presented in FIG. 5, with error bars representing standard deviation. The tested SMM formulation, Compound 6, was found to reduce the CoF of silicone tubing by 88%.

### Example 6: Reduction of Coefficient of Friction in Polyurethane Films

Polyurethane films containing 2 wt. % compound Compound 2 of the invention were prepared using a laboratory micro compounder and a heat press. Specifically, Carbothane 3585A from Lubrizol was dried in a vacuum oven at 65° C. for 4 h prior to processing, and a melt temperature of ˜230° C. was used for blending and extrusion of the polyurethane resin with SMM into a rod format as described in Example 1. Subsequently, the rods were aligned in a mold and compression molded into 130×90 mm, 1 mm thick films using a Carver Press (Model 2627-5) operated at a temperature of 210° C. and pressure of 500 psi for 5 minutes. After compression, the molds were quenched in cold water and the films promptly removed.

The coefficient of friction (CoF) of the films was measured according to the ASTM D1894 test method. The substrate chosen for the testing were 12″×12″, 1/32″ thick Silicone 60A sheets. Film samples were conditioned at 23° C.±2° C. and 50%±10% RH for 40 hours. After the conditioning period, samples were cut to 64×64 mm square specimens and taped to a sled of known weight (200 g) using double-sided tape. This sled was pulled across a plane of the silicone substrate at a speed of 150 mm/minute using an Instron Series 5565 Apparatus, and the force to initiate sled movement (static force) and to maintain motion (kinetic force) were recorded. The test was conducted in both dry and wet conditions (the substrate was sprayed with water before each wet test) at room temperature. The same side of the film was used for dry and wet tests. A new silicone substrate sheet was used for each test group. Static coefficient of friction (μₛ) was calculated by dividing the static force by the sled weight. Kinetic coefficient of friction (μₖ) was calculated by dividing the average force reading obtained during the uniform sliding of the sled across the silicone substrate surface.

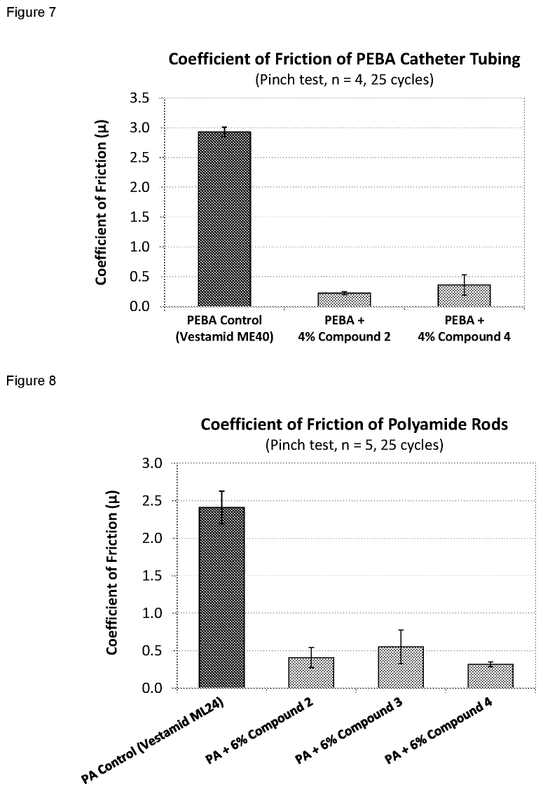

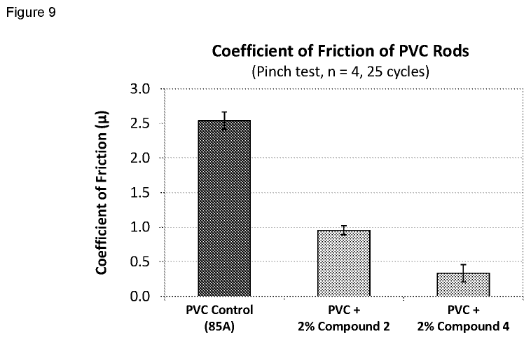

Five independent samples of unmodified polyurethane control films and SMM-modified films were tested, and the average CoF is presented in FIG. 6 with error bars representing standard deviations. The SMM formulation tested, Compound 2, was found to reduce the static CoF on polyurethane films by 37% and 68% in the dry and wet state respectively, and the kinetic CoF by 35% and 33% in dry and wet state respectively.

### Example 7: Reduction of Coefficient of Friction in PEBA Catheter Tubing

PEBA catheter tubing containing 4 wt. % Compound 2 and Compound 4 of the invention was prepared by first compounding the SMMs into PEBA (Vestamid® ME40) resin from Evonik using commercial compounding processes. Both virgin PEBA resin and compounded resin with SMM were then extruded into 8F tubing using commercial tubing extrusion processes.

The coefficient of friction (CoF) of the extruded tubing was measured by means of a pinch test using a Harland FTS 6000 tester as described in Example 1. A clamp force of 200 g was used, and 4 independent samples of unmodified PEBA control tubing and SMM-modified tubing were tested. The pinch test was repeated 25 times on each sample to confirm maintained reductions over repeat test cycles. The average CoF across 4 samples and 25 cycles is presented in FIG. 7, with error bars representing standard deviation. Both of the tested SMM formulations, Compound 2 and Compound 4, were found to reduce the CoF of the PEBA tubing by >30% (specifically by 92%, and 88% respectively).

### Example 8 Reduction of Coefficient of Friction in Polyamide Rods

Polyamide prototypes containing 6 wt. % Compounds 2, 3 and 4 of the invention were prepared using a laboratory micro-compounder as described in Example 1. Specifically, Vestamid® ML24 from Evonik was dried in a vacuum oven at 80° C. for 4 h prior to processing, and a melt temperature of ˜215° C. was used for blending and extrusion of the polyamide resin with SMM. Resulting rod prototypes were ˜3.5 mm in diameter.

The coefficient of friction (CoF) of the extruded prototypes was measured by means of a pinch test using a Harland FTS 6000 tester as described in Example 1. A clamp force of 200 g was used, and 5 independent samples of unmodified polyamide control and SMM-modified prototypes were tested. The pinch test was repeated 25 times on each sample to confirm maintained reductions over repeat test cycles. The average CoF across 5 samples and 25 cycles is presented in FIG. 8, with error bars representing standard deviation.

All three of the tested SMM formulations, Compound 2, Compound 3 and Compound 4, were found to reduce the CoF of the polyamide prototypes by >30% (specifically by 83%, 77% and 87% respectively).

### Example 9 Reduction of Coefficient of Friction in PVC Rods

PVC prototypes containing 2 wt. % Compounds 2 and 4 of the invention were prepared using a laboratory micro-compounder as described in Example 1. Specifically, 85A PVC from Cary Compounds was dried in a vacuum oven at room temperature overnight prior to processing, and a melt temperature of ˜160° C. was used for blending and extrusion of the PVC resin with SMM. Resulting rod prototypes were ˜3.6 mm in diameter.

The coefficient of friction (CoF) of the extruded prototypes was measured by means of a pinch test using a Harland FTS 6000 tester as described in Example 1. A clamp force of 200 g was used, and 4 independent samples of unmodified PVC control and SMM-modified prototypes were tested. The pinch test was repeated 25 times on each sample to confirm maintained reductions over repeat test cycles. The average CoF across 4 samples and 25 cycles is presented in FIG. 9, with error bars representing standard deviation.

Both of the tested SMM formulations, Compound 2 and Compound 4, were found to reduce the CoF of the PVC prototypes by >30% (specifically by 62%, and 87% respectively).

Obviously, numerous modifications and variations of the present invention are possible in light of the above teachings. It is therefore to be understood that within the scope of the appended claims, the invention may be practiced otherwise than as specifically described herein.

Item 1 an article, comprising:

a low friction surface comprising
an oligofluorinated additive admixed with a base polymer;

wherein a coefficient of friction of said low friction surface is reduced by at least 30% compared to a surface of the base polymer without said oligofluorinated additive.

Item 2 the article according to item 1, wherein the coefficient of friction of said low friction surface is measured by a pinch test.

Item 3 the article according to any one of items 1-2, wherein the surface is in a hydrated state.

Item 4 the article according to any one of items 1-3, wherein said article is a medical device.

Item 5 the article according to item 4, wherein said medical device is a catheter selected from the group consisting of a vascular catheter, a drainage catheter, a neurovascular catheter, an infusion catheter, a parenteral feeding catheter and a urological catheter.

Item 6 the article according to any one of items 1-5, wherein said low friction surface is an outer surface.

Item 7 the article according to any one of items 1-5, wherein said low friction surface is an inner surface.

Item 8 the article according to any one of items 1-7, wherein said low friction surface is obtained by applying a coating comprising an oligofluorinated additive and a base polymer.

Item 9 the article according to any one of items 1-8, wherein said base polymer is selected from the group consisting a polyurethane, a silicone, a polyamide, a polyester, a co-polyester, a polyether, a polyether-block-amide co-polymer, a polypropylene, a polyethylene, a polyvinylchloride, a polysulfone, a polyetherimide, a polycarbonate, a polyetheretherketone, an ethyl vinyl acetate, a polyolefin. a styrenic block copolymer, a vulcanized rubber and a mixture thereof.

Item 10 the article according to any one of items 1-8, wherein said base polymer is selected from the group consisting of a polyurethane, a silicone, a polyether block amide co-polymer, a polyamide, a polyvinyl chloride, and a mixture thereof.

Item 11 the article according to item 2, wherein a coefficient of friction, as measured by a pinch test, is reduced by at least 50% of said base polymer.

Item 12 the article according to any one of items 1-11, wherein said oligofluorinated additive is present in an amount of 0.05-15 wt. %, relative to said base polymer.

Item 13 the article according to any one of items 1-12, wherein said oligofluorinated additive is according to Formula (I):

F_{T}—[B-A]ₙ—B—F_{T} (I)
wherein
(i) A includes polypropylene oxide, polyethylene oxide, polytetramethylene oxide, hydrogenated polybutadiene, polybutadiene, hydrogenated polyisoprene, poly(diethylene glycol)adipate, (diethylene glycol-ortho phthalic anhydride) polyester, (neopentyl glycol-ortho phthalic anhydride) polyester, (1,6-hexanediol-ortho phthalic anhydride) polyester, poly(hexamethylene carbonate), poly((2,2-dimethyl)-1,3-propylene carbonate), polycarbonate polyol, poly(ethylene-co-butylene), polystyrene, polysiloxane, polydimethylsiloxane, polypropylene glycol-polyethylene glycol block copolymer, polysiloxane-polyethylene glycol block copolymer, polysiloxane-polypropylene glycol block copolymer, or other block copolymers containing block segments selected from the group of polypropylene oxide, polyethylene oxide, polytetramethylene oxide, polysiloxane, and polydimethylsiloxane;
(ii) B is a segment comprising a urethane; and
(iii) F_{T} is a polyfluoroorgano group, and
(iv) n is an integer from 1 to 10.

Item 14 the article according to item 13, wherein said base polymer is selected from the group consisting of a polyurethane, a silicone, a polyether block amide co-polymer, a polyamide, a polyvinyl chloride, and a mixture thereof.

Item 15 the article according to any one of items 1-12, wherein said oligofluorinated additive is according to Formula (II) or Formula (III):

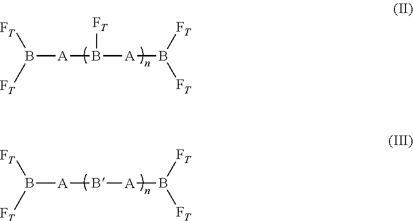

wherein
(i) A is an oligomeric segment containing an ether linkage, an ester linkage, a carbonate linkage, a polyalkylene. A includes polypropylene oxide, polyethylene oxide, polytetramethylene oxide, hydrogenated polybutadiene, polybutadiene, hydrogenated polyisoprene, poly(diethylene glycol)adipate, (diethylene glycol-ortho phthalic anhydride) polyester, (neopentyl glycol-ortho phthalic anhydride) polyester, (1,6-hexanediol-ortho phthalic anhydride) polyester, poly(hexamethylene carbonate), poly((2,2-dimethyl)-1,3-propylene carbonate), polycarbonate polyol, poly(ethylene-co-butylene), polystyrene, polysiloxane, polydimethylsiloxane, polypropylene glycol-polyethylene glycol block copolymer, polysiloxane-polyethylene glycol block copolymer, polysiloxane-polypropylene glycol block copolymer, or other block copolymers containing block segments selected from the group of polypropylene oxide, polyethylene oxide, polytetramethylene oxide, polysiloxane, and polydimethylsiloxane;
(ii) B is a segment including an isocyanurate trimer or biuret trimer; B′, when present, is a segment including a urethane;
(iii) each Fr is a polyfluoroorgano group; and
(iv) n is an integer between 0 to 10.

Item 16 the article according to any one of items 1-12, wherein said oligofluorinated additive is according to Formula (IV):

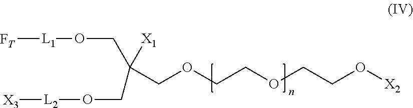

wherein
(i) each F_{T} is independently a surface-active group selected from a polydimethylsiloxane, a hydrocarbon, and a polyfluoroorgano group;
(ii) X₁ is H, CH₃, or CH₂CH₃;
(iii) each of X₂ and X₃ is independently H, CH₃, CH₂CH₃, or F_{T};
(iv) each of L₁ and L₂ is independently a bond, an oligomeric linker, or a linker with two terminal carbonyls; and
(v) n is an integer from 5 to 50.

Item 17 the article according to item 16, wherein said base polymer is selected from the group consisting of a polyurethane, a silicone, polyether block amide co-polymer, a polyamide, a polyvinyl chloride, and a mixture thereof.

Item 18 a method of reducing the coefficient of friction of a surface, said method comprising: applying a mixture of an oligofluorinated additive and a base polymer, to a surface in need thereof.

Item 19 the method according to item 18, wherein said mixture comprises of 0.05-15 wt. % of said oligofluorinated additive relative to said base polymer.

Item 20 a method of producing a medical device having a reduced coefficient of friction, said method comprising extruding, molding or coating a composition comprising an oligofluorinated additive and a base polymer.

Item 21 use of an oligofluorinated additive to lower the coefficient of friction of an polymeric article or layer, preferably by at least 30% compared the coefficient of friction of said polymeric article or layer without said oligofluorinated additive, wherein said oligofluorinated additive is admixed with the base polymer of the polymeric article or layer.

What is claimed is:

1. An article, comprising:
a low friction surface comprising
an oligofluorinated additive admixed with a base polymer;
wherein a coefficient of friction of said low friction surface is reduced by at least 30% compared to a surface of the base polymer without said oligofluorinated additive.

2. The article according to claim 1, wherein the coefficient of friction of said low friction surface is measured by a pinch test.

3. The article according to claim 1, wherein the surface is in a hydrated state.

4. The article according to claim 1, wherein said article is a medical device.

5. The article according to claim 4, wherein said medical device is a catheter selected from the group consisting of a vascular catheter, a drainage catheter, a neurovascular catheter, an infusion catheter, a parenteral feeding catheter and a urological catheter.

6. The article according to claim 1, wherein said low friction surface is an outer surface.

7. The article according to claim 1, wherein said low friction surface is an inner surface.

8. The article according to claim 1, wherein said low friction surface is obtained by applying a coating comprising an oligofluorinated additive and a base polymer.

9. The article according to claim 1, wherein said base polymer is selected from the group consisting a polyurethane, a silicone, a polyamide, a polyester, a co-polyester, a polyether, a polyether-block-amide co-polymer, a polypropylene, a polyethylene, a polyvinylchloride, a polysulfone, a polyetherimide, a polycarbonate, a polyetheretherketone, an ethyl vinyl acetate, a polyolefin, a styrenic block copolymer, a vulcanized rubber and a mixture thereof.

10. The article according to claim 1, wherein said base polymer is selected from the group consisting of a polyurethane, a silicone, a polyether block amide co-polymer, a polyamide, a polyvinyl chloride, and a mixture thereof.

11. The article according to claim 2, wherein a coefficient of friction, as measured by a pinch test, is reduced by at least 50% of said base polymer.

12. The article according to claim 1, wherein said oligofluorinated additive is present in an amount of 0.05-15 wt. %, relative to said base polymer.

13. The article according to claim 1, wherein said oligofluorinated additive is according to Formula (I):
F_{T}—[B-A]ₙ—B—F_{T} (I)
wherein
(i) A includes polypropylene oxide, polyethylene oxide, polytetramethylene oxide, hydrogenated polybutadiene, polybutadiene, hydrogenated polyisoprene, poly(diethylene glycol)adipate, (diethylene glycol-ortho phthalic anhydride) polyester, (neopentyl glycol-ortho phthalic anhydride) polyester, (1,6-hexanediol-ortho phthalic anhydride) polyester, poly(hexamethylene carbonate), poly((2,2-dimethyl)-1,3-propylene carbonate), polycarbonate polyol, poly(ethylene-co-butylene), polystyrene, polysiloxane, polydimethylsiloxane, polypropylene glycol-polyethylene glycol block copolymer, polysiloxane-polyethylene glycol block copolymer, polysiloxane-polypropylene glycol block copolymer, or other block copolymers containing block segments selected from the group of polypropylene oxide, polyethylene oxide, polytetramethylene oxide, polysiloxane, and polydimethylsiloxane;
(ii) B is a segment comprising a urethane; and
(iii) F_{T} is a polyfluoroorgano group, and
(iv) n is an integer from 1 to 10.

14. The article according to claim 13, wherein said base polymer is selected from the group consisting of a polyurethane, a silicone, a polyether block amide co-polymer, a polyamide, a polyvinyl chloride, and a mixture thereof.

15. The article according to claim 1, wherein said oligofluorinated additive is according to Formula (II) or Formula (III): 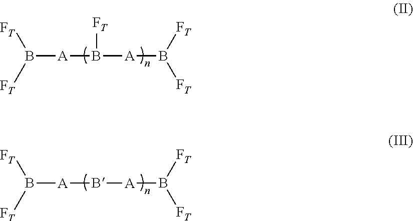
wherein
(i) A is an oligomeric segment containing an ether linkage, an ester linkage, a carbonate linkage, a polyalkylene. A includes polypropylene oxide, polyethylene oxide, polytetramethylene oxide, hydrogenated polybutadiene, polybutadiene, hydrogenated polyisoprene, poly(diethylene glycol)adipate, (diethylene glycol-ortho phthalic anhydride) polyester, (neopentyl glycol-ortho phthalic anhydride) polyester, (1,6-hexanediol-ortho phthalic anhydride) polyester, poly(hexamethylene carbonate), poly((2,2-dimethyl)-1,3-propylene carbonate), polycarbonate polyol, poly(ethylene-co-butylene), polystyrene, polysiloxane, polydimethylsiloxane, polypropylene glycol-polyethylene glycol block copolymer, polysiloxane-polyethylene glycol block copolymer, polysiloxane-polypropylene glycol block copolymer, or other block copolymers containing block segments selected from the group of polypropylene oxide, polyethylene oxide, polytetramethylene oxide, polysiloxane, and polydimethylsiloxane;
(ii) B is a segment including an isocyanurate trimer or biuret trimer; B′, when present, is a segment including a urethane;
(iii) each F_{T} is a polyfluoroorgano group; and
(iv) n is an integer between 0 to 10.

16. The article according to claim 1, wherein said oligofluorinated additive is according to Formula (IV): 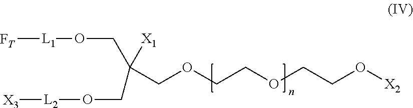
wherein
(i) each F_{T} is independently a surface-active group selected from a polydimethylsiloxane, a hydrocarbon, and a polyfluoroorgano group;
(ii) X₁ is H, CH₃, or CH₂CH₃;
(iii) each of X₂ and X₃ is independently H, CH₃, CH₂CH₃, or F_{T};
(iv) each of L₁ and L₂ is independently a bond, an oligomeric linker, or a linker with two terminal carbonyls; and
(v) n is an integer from 5 to 50.

17. The article according to claim 16, wherein said base polymer is selected from the group consisting of a polyurethane, a silicone, a polyether block amide co-polymer, a polyamide, a polyvinyl chloride, and a mixture thereof.

18. A method of reducing the coefficient of friction of a surface, said method comprising:
applying a mixture of an oligofluorinated additive and a base polymer, to a surface in need thereof.

19. The method according to claim 18, wherein said mixture comprises of 0.05-15 wt. % of said oligofluorinated additive relative to said base polymer.

20. A method of producing a medical device having a reduced coefficient of friction, said method comprising extruding, molding or coating a composition comprising an oligofluorinated additive and a base polymer.
